(19) United States
(12) Patent Application Publication
(10) Pub. No.: US 2023/0235354 A1
(43) Pub. Date: Jul. 27, 2023

(54) ENHANCEMENT OF THE PRODUCTION OF ADENOIDVIRUS-BASED GENETRANSFER VECTORS

(71) Applicant: Greffex, Inc., Houston, TX (US)

(72) Inventor: Uwe D. STAERZ, Denver, CO (US)

(21) Appl. No.: 17/995,128

(22) PCT Filed: Mar. 29, 2021
(86) PCT No.: PCT/US2021/024578

§ 371 Date: Sep. 30, 2022

Related U.S. Application Data
(60) Provisional application No. 63/001,758, filed on Mar. 30, 2020.

(51) Int. Cl. C12N 15/86; C12N 15/113

(52) U.S. Cl. CPC C12N 15/86; C12N 15/113; C12N 2330/51; C12N 2710/10041; C12N 2710/10052; C12N 2310/11

(57) ABSTRACT

In one aspect, the embodiments disclosed herein relate to the production of fully-deleted adenovirus-based gen delivery vectors packaged without the use of an adenoviral helper virus, and more particularly in their use in the transfer of genes and the expression of proteins, vaccine development, and cell engineering. In another aspect, the production of adenoviral vectors deleted of all adenoviral genes is described that carry genes of interest with detrimental or toxic activities to eukaryotic cells.

### FIELD

The embodiments disclosed herein relate to the production of fully deleted adenovirus-based vector delivering genes detrimental or toxic for eukaryotic cells packaged without helper adenovirus, and more particularly, to their use in gene therapy for gene and protein expression, vaccination, and modification of cells and tissues BACKGROUND

Among the most commonly used vectors for the delivery of genetic material into human cells are the adenoviruses. Adenoviruses have been isolated from a large number of different species, and more than 100 different serotypes have been reported. The overall organization of the adenoviral genome is conserved among serotypes, such that specific functions are similarly positioned. Adenoviruses of different serotypes have been completely sequenced and their genome sequences are publicly available. Many adults have been exposed to the human adenovirus of the serotype 5 (Ad5) that has formed the basis of many gene transfer vectors.

The Ad5 genome is a linear, non-segmented, double stranded DNA, approximately 35 kbp (size varies from group to group) which has the theoretical capacity to encode 30-40 genes. The Ad5 genome is flanked on both sides by inverted terminal repeat sequences (LITR and RITR), which are essential to replication of adenoviruses. The infectious cycle of adenoviruses, such as Ad5, is divided into an early and a late phase. In the early phase, the virus is uncoated and genome transported to the nucleus, after which the early gene regions (E), E1, E2, E3 and E4 become transcriptionally active. E1 contains two regions named E1A and E1 B. The E1A region (sometimes referred to as immediate early region) encodes two major proteins that are involved in modification of the host-cell cycle and activation of the other viral transcription regions. The E1 B region encodes two major proteins, 19K and 55K, that prevent, via different routes, the induction of apoptosis resulting from the activity of the E A proteins. In addition, the E1 B-55K protein is required in the late phase for selective viral mRNA transport and inhibition of host protein expression. E2 is also divided in E2A and E2B region that together encode three proteins. DNA binding protein, viral polymerase and pre-terminal protein, all involved in the replication of the viral genome. The E3 region is not required for replication in vitro, but enocdes several proteins that subvert the host defense mechanism toward viral infection. The E4 region encodes at least six proteins involved in several distinct functions related to viral mRNA splicing and transport, host cell mRNA transport, viral and cellular transcription and transformation.

The late proteins necessary for formation of the viral capsids and packaging of the viral genome, are all generated from the major late transcription unit that becomes fully active after the onset of viral DNA replication. A complex process of differentiated splicing and polyadenylation gives rise to more than 15 mRNA species that share a tripartite leader sequence. The early proteins E1 B-55K and E4-Orf3 and Orf6 play a pivotal role in the regulation of late mRNA processing and transport from the nucleus.

Packaging of newly formed adenoviral genomes in pre-formed capsids is mediated by at least two adenoviral proteins, the late 52/55k and an intermediate protein 1Va2, through interaction with the viral packaging signal (Ψ) located at the left end of the Ad5 genome. A second intermediate protein pIX is part of the capsid and is known to stabilize the hexon-hexon interactions. In addition, pIX has been described to transactivate TATA-containing promoters like the EIA promoter and the major late promoter (MLP).

Adenovirus-Based Vectors and Adenoviral Packaging Cell Lines

Adenovirus-based vectors have been used as a means to achieve high level gene transfer into various cell types, as vaccine delivery vehicles, for gene transfer into tissue transplants, for gene therapy, and to express recombinant proteins in cell lines and tissues that are otherwise difficult to transfect with high efficiency. Current systems for packaging Adenovirus-based vectors consist of a host cell and a source of the adenoviral late genes. The current known host cell lines, including the 293, OBI, and PERC.6 cells, express only early (nonstructural) adenovirus genes, not the late adenoviral (structural) genes needed for packaging. The adenoviral late genes have previously been provided either by the adenoviral vectors themselves in cis or by a helper adenoviral virus in trans. The adenoviral vectors that provide the genes themself necessary for their encapsidation carry minimally modified adenoviral genomes principally deleted by gene of the E 1 and some cases also the E3 and other adenoviral regions.

More recently, “gutless” adenoviral vectors-vectors that are devoid of all viral protein-coding DNA sequences have been developed. The gutless adenoviral vectors contain only the ends of the viral genome (LITR and RITR), genes of interest, such as therapeutic genes, and the normal packaging recognition signal (Ψ), which allows this genome to be selectively packaged. However, to propagate the gutless adenoviral vector requires a helper adenovirus that contains the adenoviral genes required for replication and virion assembly as well as LITR, RITR, and Ψ. While this helper virus-dependent system allows the introduction of up to about 35 kb of foreign DNA, the helper virus contaminates the preparations of gutless adenoviral vectors using this approach Contaminating replication competent helper viruses pose serious problems for gene therapy, vaccine, and transplant applications both because of the replication competent virus and because of the host's immune response to the adenoviral genes in the helper virus. One approach to decrease helper contamination in this helper virus-dependent vector system has been to introduce a conditional gene defect in the packaging recognition signal (T′) making it less likely that its DNA is packaged into a virion. Gutless Adenoviral vectors produced in such systems still have significant contamination with helper virus. Being able to produce gutless adenoviral gene transfer vectors without helper virus contamination would eliminate helper virus contamination resulting in reduced vector toxicity and prolonged gene expression in human subjects and animals.

It is believed that adenoviral genes especially adenoviral late genes carried in minimally modified adenoviral vectors or in adenoviral helper viruses: 1) contribute to the inflammatory response seen after adenoviral mediated gene therapy, 2) decrease the immune response towards the gene of interest in vaccine applications, 3) interfere with normal cellular functions, and 4) result in protein contaminants in protein expression applications. Further, they occupy space in minimally modified adenoviral vectors that could be beneficially be used for carrying other genetic information. Remarkable progress has been made with adenoviral vectors in the last decade, but serious shortcomings continue to challenge their use.

Adenovirus Vectors for Gene Therapy and Protein Expression

Gene delivery or gene therapy is a promising method for the treatment of acquired and inherited diseases. An ever-expanding array of genes for which abnormal expression is associated with life-threatening human diseases are being cloned and identified. The ability to express such cloned genes in humans will ultimately permit the prevention and/or cure of many important human diseases, diseases for which current therapies are either inadequate or nonexistent.

Unfortunately, however, gene therapy protocols described to date have been plagued by a variety of problems, including in particular the short period of gene expression from the vector and the inability to effectively re-administer the same vector a second time, both of which may be caused by the host immune response against antigens associated with the vector and its therapeutic payload. Tissues that have incorporated the viral and/or therapeutic genes are initially attacked by the host's cellular immune response, mediated by CD8+ cytotoxic T cells as well as CD4+ helper T cells, which dramatically limits the persistence of gene expression from the vectors. Moreover, the host's humoral immune response mediated by the CD4+ T cells further limits the effectiveness of current gene therapy protocols by inhibiting the successful re-administration of the same vector.

For example, following an initial administration of adenoviral vector, serotype-specific antibodies are generated against epitopes of the major viral capsid proteins, namely the penton, hexon and fiber. Given that such capsid proteins are the means by which the adenovirus attaches itself to a cell and subsequently infects the cell, such antibodies are then able to block or “neutralize” reinfection of a cell by the same serotype of adenovirus or adenoviral vector. This may necessitate using a different serotype of Adenovirus in order to administer one or more subsequent doses of exogenous therapeutic DNA in the context of gene therapy and vaccines. In addition, both therapeutic and viral gene products are expressed on target cells when using minimally modified adenoviral vectors or adenoviral helper virus contaminated adenoviral vector preparations. These antigens can be recognized by cellular immune responses leading to the destruction of the transduced cells or tissues and thus the beneficial effect of gene therapy and vaccination may be negated. As a result of these immune-related obstacles the widespread use of minimally modified viral vectors has been stymied.

At least 53 different forms of human adenovirus and in addition numerous animal adenoviruses have been characterized. The principal discriminating factor among these viruses is the humoral immune (i.e. antibody) response to the capsid hexon protein (encoded by various alleles of the L3 gene). In fact, the majority of variation among the different hexon proteins occurs in three “hyper”-variable regions; the humoral immune response to Adenoviruses is centered on these hypervariable regions. Other structures, such as the fiber proteins on the adenoviral surface can also be recognized by the humoral immune systems. The interference of humoral immune responses with the activity of minimally modified adenoviral vectors can therefore be mitigate by switching adenoviral serotypes between each application. Late adenoviral genes show less variability and therefore T cell responses induced by minimally modified adenoviral vectors or adenoviral helper viruses cannot be avoided by switching the adenoviral serotype of the vectors.

Human populations have been exposed to natural adenovirus infections of certain adenoviral serotypes. Therefore, these subjects carry humoral and cellular immune responses directed genes expressed by these adenoviruses and adenoviral vectors based on adenoviruses of these serotypes. Two advances have sought to overcome the problems. They are the use of “gutless” (fully deleted) adenoviral vectors and the use adenoviral vectors based on rare or animal adenoviruses expressing rare or animal serotypes. While the use of “gutless” adenoviral vectors removes the adenoviral genes, such as L3, from the therapeutic vector, the propagation of these “gutless” adenoviral vectors requires the presence of helper adenoviruses that still carry the adenoviral genes. These helper viruses are significant contaminants in the preparations of “gutless” adenoviral vectors. The use of minimally modified adenoviral vectors based on rare or animals serotypes may avoid the problem of pre-existing humoral immunity and possibly to a lesser extent pre-existing cellular immunity in that subjects who have been previously been exposed to an adenovirus of a given serotype. Still, as the minimally modified adenoviral vectors express adenoviral genes including the highly immunogenic L3, they may induce potent humoral and cellular immune responses to these adenoviral genes. Therefore, repeated applications of a minimally modified adenoviral vector of a given serotype will not be possible.

Adenoviruses as Vaccine Vectors

Adenoviral vectors have transitioned from tools for gene replacement therapy to bona fide vaccine delivery vehicles. They are attractive vaccine vectors as they induce both innate and adaptive immune responses in mammalian hosts. Adenoviral vectors have been tested to deliver as subunit vaccine systems for numerous infections infectious diseases, such as malaria, tuberculosis, Ebola and HIV-1. Additionally they have been explored as vaccines against different tumor associated antigens. Thus far most adenovirally vectored vaccines have been constructed as minimally modified adenoviral vectors of human and animal serotypes.

The dynamics of adenoviral gene expression have made the design of adenoviral packaging systems difficult: expression of the adenoviral early functional transcription region (E1A) gene induces expression of the adenoviral late genes (structural, immunogenic genes), which in turn kills the cell.

Accordingly, a host cell that constitutively expresses the adenoviral early genes cannot carry the “wildtype” adenoviral late cistron. Previous host cells for propagating adenoviral vectors are not bona fide “packaging” cells. Specifically, the 293. QBI and PERC 6 cells express only early (non-structural) adenoviral genes, not the adenoviral late genes needed for packaging. Adenoviral late also early genes have to be provided. They previously been provided either by the minimally modified adenoviral vector in cis or by a helper adenovirus in trans.

The adenoviral genes found in minimally modified adenoviral vectors or in contaminating helper adenoviruses contribute to inflammatory and immune responses to the adenoviral vector preparation, decrease the immune response to a gene of interest of an adenoviral based vaccine; interferes with normal cellular functions; and to contamination in adenovirally based protein expression.

It may be beneficial to employ potent and broadly active promoters, such as viral promoters, for the expression of the gene of interest. This may be necessary to assure effective gene therapy or potent immunogenicity. Gene transfer vectors may however carry genes of interest that are detrimental to or toxic for host cells used for packaging of the adenoviral vectors. Therefore their expression may interfere with an efficient encapsidation of adenoviral vectors, both minimally modified and “gutted” ones. These genes may be toxic or detrimental by design to, for instance, remove cancerous cells after transduction, or they may constitute a bacterial or viral gene, though detrimental for or toxic to the host cell at high concentration, to which protective immune responses can be raised, such as the Ebola glycoprotein. Therefore it may be necessary to downregulate the expression of the gene of interest during packaging. The described invention addresses this problem. It provides systems and methods for the production of minimally modified adenoviral vectors or “gutted” (fully deleted) adenoviral vectors without participation of an adenoviral helper virus that carry genes of interest with functions detrimental or toxic for host cells used for encapsidation of these vectors Uses of such vectors will be described.

### SUMMARY

The embodiments disclosed herein relate to the construction and production of fully deleted adenoviral vectors packaged with a helper virus. They also relate to the production of minimally modified adenoviral vectors and fully deleted adenoviral vectors that carry genes of interest with functions and activities detrimental or toxic for host cells used for the production or encapsidation of these vectors. They relate to these vectors used in gene therapy, vaccination, cancer therapy, and immune suppressive therapy.

According to aspects illustrated herein, there is provided a system that includes (a) an adenovirus host cell for packaging of adenoviral vectors; (b) a fully-deleted adenoviral vector module construct; (c) a packaging expression plasmid carrying genes able to encapsidate adenoviral vector modules into adenoviral capsids; (d) an expression construct, either on separate expression vector or incorporated into the packaging expression plasmid able to inhibit expression of the gene of interest on the adenoviral vector module; (e) and alternatively sets of short inhibitory RNA or DNA fragments that bind to the gene of interest on adenoviral vector module, able to inhibit expression of the gene of interest. The adenoviral vector module either a minimally modified of a fully deleted adenoviral vector is co-transfected optionally with the packaging expression plasmid, the expression construct or sets of RNA or DNA fragments able to inhibit expression of the gene of interest on the adenoviral vector module.

The packaging expression vector and the expression construct inhibiting the gene of interest are themselves unable to be encapsidated. The adenoviral vector module by itself is unable to replicate. According to aspects illustrated herein, there is disclosed a method for the propagation of adenoviral vectors with detrimental of toxic genes includes (a) providing an adenovirus packaging cell; (b) transfecting an adenoviral vector module deleted of all adenoviral genes into the packaging cell line; (c) transfecting a packaging expression plasmid into the packaging cell, wherein the fully deleted adenoviral vector module and the packaging expression plasmid transfect the packaging cell resulting in the encapsidation of the fully deleted adenoviral vector module in an adenoviral capsid independent of a helper virus; (d) transfecting of an inhibitory expression plasmid that codes for the expression of anti-sense RNA of the gene of interest found on fully the deleted adenoviral vector module into the packaging cell, wherein the expression of the gene of interest on the fully deleted adenovirus vector module is inhibited; (e) transfecting a fully deleted adenoviral vector module, a packaging expression plasmid and an inhibitory expression plasmid into packaging cells to inhibit expression of the gene of interest du ring vector encapsidation and to improve packaging of the fully adenoviral vector module into adenoviral capsids; (f) transfecting short inhibitory RNA or DNA fragments that bind to the gene of interest found on the fully deleted adenoviral vector module, wherein the expression of the gene of interest on the fully deleted vector module is inhibited; (g) transfecting a fully deleted adenoviral vector module, a packaging expression plasmid and short inhibitory RNA or DNA fragments into packaging cells to inhibit expression of the gene of interest du ring vector encapsidation and to improve packaging of the fully adenoviral vector module into adenoviral capsids.

According to aspects illustrated herein, there is disclosed method for the propagation of adenoviral vectors with detrimental of toxic genes includes (a) providing an adenovirus packaging cell, (b) introducing into the packaging cells a minimally modified adenoviral vector; (c) transfecting of an inhibitory expression plasmid that codes for the expression of anti-sense RNA of the gene of interest found on fully the deleted adenoviral vector module into the packaging cell, wherein the expression of the gene of interest on the fully deleted adenovirus vector module is inhibited. (d) transfecting a fully deleted adenoviral vector module, a packaging expression plasmid and an inhibitory expression plasmid into packaging cells to inhibit expression of the gene of interest during vector encapsidation and to improve packaging of the fully adenoviral vector module into adenoviral capsids; (e) transfecting short inhibitory RNA or DNA fragments that bind to the gene of interest found on the fully deleted adenoviral vector module, wherein the expression of the gene of interest on the fully deleted vector module is inhibited; (g) transfecting a fully deleted adenoviral vector module, a packaging expression plasmid and short inhibitory RNA or DNA fragments into packaging cells to inhibit expression of the gene of interest during vector encapsidation and to improve packaging of the fully adenoviral vector module into adenoviral capsids.

In an embodiment, a target cell is transduced with the encapsidated fully deleted adenoviral vector for the treatment of a condition, disease or disorder. In an embodiment, the encapsidated fully deleted adenoviral vector is injected into a human subject so that the expression of the gene of interest exerts curative functions or induces immune responses. In an embodiment, the encapsidated fully deleted adenoviral vector is used to transduce target cells or target tissues to modify their activity or to modify the response of other cells and tissue components to the target cells or target tissues.

In some embodiments, gene transfer vectors of the present disclosure are useful in a method of treating cancer. In some embodiments, gene transfer vectors of the present disclosure are useful in a method of treating skin disorders. In some embodiments, gene transfer vectors of the present disclosure are useful in a method of treating vascular disease. In some embodiments, gene transfer vectors of the present disclosure are useful in a method of treating cardiac disease. In some embodiments, gene transfer vectors of the present disclosure are useful in a method of treating cardiac disease. In some embodiments, gene transfer vectors are useful in a method of treating an autoimmune disease. In some embodiments, gene transfer vectors of the present disclosure are useful in a method of treating a parasitic infection. In some embodiments, gene transfer vectors of the present disclosure are useful in a method of treating a viral infection. In some embodiments, gene transfer vectors of the present disclosure are useful in a method of treating a bacterial infection.

In some embodiments, gene transfer vectors of the present disclosure are useful in a method of treating a yeast infection. In some embodiments, gene transfer vectors of the present disclosure are useful in a method of treating a neurological disease. In some embodiments, gene transfer vectors of the present disclosure are useful in a method of treating a hereditary disease.

In some embodiments, an encapsidated adenoviral vector produced by a method of the present disclosure is used as a gene delivery vector for protein expression. In some embodiments, an encapsidated adenoviral vector produced by a method of the present disclosure is used in developing and manufacturing vaccines. In some embodiments, an encapsidated adenoviral vector produced by a method of the present disclosure is used as a gene delivery vector for immune suppressive therapy. In an embodiment, a target cell is transduced with an encapsidated adenoviral vector previously produced.

Other objects and features will be in part apparent and in part pointed out hereinafter.

### DESCRIPTION OF THE DRAWINGS

FIG. 1 is a diagrammatic representation showing an embodiment of a design of a fully deleted adenoviral vector module.

FIG. 2 is a diagrammatic representation showing an embodiment of the design of packaging expression plasmids with and without expression cassettes able to inhibit expression of genes of interest on the adenoviral vector module.

FIG. 3 is a diagrammatic representation showing an embodiment of a design of a minimally modified adenoviral vector.

FIG. 4. is a diagrammatic representation showing an embodiment of a design of an inhibitory expression plasmid carrying an expression cassette or expression cassettes able to inhibit expression of a gene or genes of interest found on the adenoviral vector module.

FIG. 5 is a diagrammatic representation showing an embodiment of a method for the encapsidation by co-transfection of a fully deleted adenoviral vector module with a packaging expression plasmid.

FIG. 6 is a diagrammatic representation showing an embodiment of a method for the encapsidation by co-transfection of a fully deleted adenoviral vector module with a packaging plasmid and an expression plasmid able to inhibit expression of the gene of interest on the adenoviral vector module.

### DETAILED DESCRIPTION

The present disclosure provides, among other things, fully deleted adenoviral vector modules, packaging expression plasmids and adenovirus packaging cells for propagating fully-deleted adenovirus-based gene transfer vectors packaged without adenoviral helper virus. The gene transfervectors as minimally modified and fully deleted vectors find use in gene therapy for gene and protein expression, vaccine development, cell and tissue manipulation and immunesuppressive therapy. Any subtype, mixture of subtypes, or chimeric Adenovirus may be used as the source of DNA for generation of an adenoviral gene transfer vectors. In an embodiment, the source of DNA is from human serotype 5.

In the systems disclosed herein a fully deleted adenoviral vector is co-transfected with a packaging expression plasmid. Expression cassettes able to interfere with genes of interest are found on the packaging expression vector or on a separate expression vector that is co-transfected as well. Alternative RNA or DNA fragments inhibitory to the expression of the gene of interest on the adenoviral vector module are co-transfected. In another system disclosed herein a minimally modified adenoviral vector is exposed in the packaging cell to an expression vector expressing genes able to interfere with genes of interest found on the packaging expression vector or to RNA or DNA fragments inhibitory to the expression of the gene of interest on the adenoviral vector defined otherwise, all technical and scientific terms used herein have the same meaning as commonly understood by one of ordinary skill in the art, to which this disclosure belongs. Generally, the nomenclature used herein and the laboratory procedures in cell culture, molecular genetics, and nucleic acid chemistry and hybridization described below are those wellwell known and commonly employed in the art. Standard techniques are used for recombinant nucleic acid methods, polynucleotide synthesis, and microbial culture and transformation (e g., electroporation, lipofection). Generally, enzymatic reactions and purification steps are performed according to the manufacturer's specifications. The techniques and procedures are generally performed according to conventional methods in the art and various general references (see generally, Sambrook eta!. Molecular Cloning: A Labaratory Manual, 2d ed. (1989) Cold Spring Labaratory Press, Cold Spring Harbor, N.Y., which is incorporated herein by reference) which are provided throughout this document. Units, prefixes, and symbols may be denoted in their SI accepted form. Unless otherwise indicated, nucleic acids are written left to right in 5′ to 3′ orientation; amino acid sequences are written left to right in amino to carboxyl orientation, respectively. Numeric ranges are inclusive of the numbers defining the range and include each integer within the defined range. Amino acids may be referred to herein by either their commonly known three letter symbols or by the one-letter symbols recommended by the IUPAC-IUB Biochemical nomenclature Commission. Nucleotides, likewise, may be referred to by their commonly accepted single-letter codes Unless otherwise provided for, software, electrical, and electronics terms as used herein are as defined in The New IEEE Standard Dictionary of Electrical and Electronics Terms (5′h edition, 1993). As employed throughout the disclosure, the following terms, unless otherwise indicated, shall be understood to have the following meanings and are more fully defined by reference to the specification as a whole: The terms “Adenovirus” and “Adenoviral particle” as used herein include any and all viruses that may be categorized as an adenovirus, including any adenovirus that infects a human or an animal, including all groups, subgroups, and serotypes. Thus, as used herein, “Adenovirus” and “Adenovirus particle” refer to the virus itself or derivatives thereof and cover all serotypes and subtypes and both naturally occurring and recombinant forms. In one embodiment, such adenoviruses infect human cells.

Such adenoviruses may be wildtype or may be modified in various ways known in the art or as disclosed herein. Such modifications include modifications to the Adenovirus genome that is packaged in the particle in order to make an infectious virus Such modifications include deletions known in the art, such as deletions in one or more of the Ela, E1b, E2a, E2b, E3, or E4 coding regions. An “adenovirus packaging cell” is a cell that is able to package adenoviral genomes or modified genomes to produce viral particles. It can provide a missing gene product or its equivalent. Thus, packaging cells can provide complementing functions for the genes deleted in an adenoviral genome and are able to package the adenoviral genomes into the adenovirus particle. The production of such particles requires that the genome be replicated and that those proteins necessary for assembling an infectious virus are produced. The particles also can require certain proteins necessary for the maturation of the viral particle. Such proteins can be provided by an minimally modified vector, a packaging expression plasmid or by the packaging cell. Exemplary host cells that may be used to make a packaging cell line according to the present invention include, but are not limited to A549, Hela, MRC5, W138, CHO cells, Vera cells, human embryonic retinal cells, human embryonal kidney cells or any eukaryotic cells, as long as the host cells are permissive for growth of Adenovirus. Some host cell lines include adipocytes, chondrocytes, epithelial, fibroblasts, glioblastoma, hepatocytes, keratinocytes, leukemia, lymphoblastoid, monocytes, macrophages, myoblasts, and neurons. Other cell types include, but are not limited to, cells derived from primary cell cultures, e.g., human primary prostate cells, human embryonic retinal cells, human stem cells. Eukaryotic diploid and aneuploid cell lines are included within the scope of the invention. The packaging cell must be one that is capable of expressing the products of the adenoviral vector and/or packaging expression plasmid and the inhibitory expression cassettes and/or vectors at the appropriate level for those products in order to generate a high titer stock of recombinant gene transfer vectors.

By “antigen” is meant a molecule which contains one or more epitopes that will stimulate a host's immune system to make a cellular antigen-specific immune response, or a humoral antibody response. Thus, antigens include proteins, polypeptides, antigenic protein fragments, oligo saccharides, polysaccharides, and the like. Furthermore, the antigen can be derived from any known virus, bacterium, parasite, plants, protozoans, or fungus, and can be a whole organism. The term also includes tumor antigens. Similarly, an oligonucleotide or polynucleotide which expresses an antigen, such as in DNA immunization applications, is also included in the definition of antigen. Synthetic antigens are also included, for example, poly-epitopes, flanking epitopes, and other recombinant or synthetically derived antigens (Bergmann eta!. (1993) Eur. J. Immunol. 23:2777 2781, Bergmann eta!. (1996) J. Immunol 157.3242 3249; Suhrbier, A. (1997) Immunol. and Cell Bioi. 75:402 408; Gardner eta!. (1998) 12th World AIDS Conference, Geneva, Switzerland, Jun. 28-Jul. 3, 1998).

A “coding sequence” or a sequence which “encodes” a selected polypeptide, is a nucleic acid molecule which is transcribed (in the case of DNA) and translated (in the case of mRNA) into a polypeptide in vivo when placed under the control of appropriate regulatory sequences (or “control elements”). The boundaries of the coding sequence are determined by a start codon at the 5′ (amino) terminus and a translation stop codon at the 3′ (carboxy) terminus. A transcription sequence may be located 3′ to the coding sequence. Transcription and translation of coding sequences are typically regulated by “control elements,” including, but not limited to, transcription promoters, transcription enhancer elements. Shine and Delagamo sequences, transcription termination signals, polyadenylation sequences (located 3′ to the translation stop codon), sequences for optimization of initiation of translation (located 5′ to the coding sequence), and translation termination sequences.

The term “construct” refers to at least one of a fully deleted adenoviral vector module of the present invention, a packaging expression vector of the present invention, or an inhibitory expression cassette located on an expression plasmid. The term “delete” or “deleted” as used herein refers to expunging, erasing, or removing.

The term “E1 region” as used herein refers to a group of genes present in the adenovirus genome. These genes, such as, but not limited to, E1A and E1B, are expressed in the early phase of virus replication and activate the expression of the other viral genes. In an embodiment, an Adenoviral packaging cell line of the present disclosure includes all coding sequences that make up the E1 region.

In an embodiment, an adenoviral packaging cell line of the present disclosure includes some coding sequences that make up the E1 region (for example, E1A or E1B) As used herein, the term “E1A” refers to all gene products of the Adenovirus E1A region, including expression products of the two major RNAs: 13S and 12S. These are translated into polypeptides of 289 and 243 amino acids, respectively. These two proteins differ by 46 amino acids, which are spliced from the 12S mRNA, as described in Chow et al. (1980) Cold Spring Harb Symp Quant Bioi. 44 Pt 1:40114; and Chow et al. (1979) J. Mol. Biol. 134(2): 265 303, herein specifically incorporated by reference. For the purposes of the invention, the packaging cell line may express the 289 polypeptide, the 243 polypeptide, or both the 289 and the 243 polypeptide. The term E1A is also used herein with reference to partial and variant E1A coding sequences. As used herein, the term “E1 B” refers to all gene products of the Adenovirus E1 B region, including the 3 major polypeptides of 19 kd and 55 kd. The E 1 8 19 kd and 55 kd proteins are important in cell transformation. For the purposes of the invention, the packaging cell line may express the 19 Kd polypeptide, the 55 Kd polypeptide, or both the 19 and the 55 Kd polypeptide. The term E1 B is also used herein with reference to partial and variant E1B coding sequences.

The term “E2” as used herein refers to a cistron with at least 3 ORFs all of which are involved in DNA replication, including a polymerase. The E2 late promoter of adenovirus has been described, for example, by Swaminathan, S., and Thimmapaya, 8. (1995) Gurr. Top. Microbial. Immunol., 199, 177-194. In the Adenoviral system, the E2 late promoter, together with the E2 early promoter, has the function of controlling the adenoviral E2 region and/or genes E2A and E2B. In this case, the synthesis of the E2 mRNA takes places initially starting out from the E2 early promoter. Approximately five to seven hours after the infection of a cell, a switch-over to the E2 later promoter takes place.

The term “E3 region” as used herein refers to a group of genes that are present in the Adenovirus genome and are expressed in the early phase of the virus replication cycle. These genes express proteins that interact with the host immune system. They are not necessary for virus replication in vitro, and therefore may be deleted in Adenovirus vectors.

The term “E4 region” as used herein refers to a group of genes that are present in the Adenovirus genome next to the right ITR, and are expressed in the early phase of the virus replication cycle. The E4 region includes at least 7 ORFs. The products of the E4 region promote viral gene expression and replication, interact with host cell components, and participate in lytic infection and oncogenesis.

The term “expression” refers to the transcription and/or translation of an endogenous gene, transgene or coding region in a cell. The terms “fully-deleted adenoviral. “gutless”, “gutted”, “mini”, “fully-deleted”, or “pseudo” vectors as used herein refers to a linear, double-stranded DNA molecule with inverted terminal repeats (ITRs) separated by approximately 26 to 37 kb, the viral packaging signal (Ψ), and at least one DNA insert (all or a fragment of at least one gene of interest (GOI)) which comprises a gene sequence encoding a protein of interest. The gene sequence can be regulatable. Regulation of gene expression can be accomplished by one of 1) alteration of gene structure: site-specific recombinases (e.g., Cre based on the Cre-loxP system) can activate gene expression by removing inserted sequences between the promoter and the gene; 2) changes in transcription: either by induction (covered) or by relief of inhibition; 3) changes in mRNA stability, by specific sequences incorporated in the mRNA or by siRNA; and 4) changes in translation, by sequences in the mRNA.

No viral coding genes are comprised in the fully deleted adenoviral vector module. Fully deleted adenoviral vector modules are also called “high-capacity” adenoviral vectors because they can accommodate up to 36 kilobases of “foreign” DNA. As vector capsids package efficiently only DNA the size of 75-105% of the whole adenovirus genome, and as therapeutic expression cassettes usually do not add up to 36 kb, there is a need to use “stuffer” DNA in order to complete the genome size for encapsidation. Earlier fully deleted adenoviral vectors are referred to as “helper dependent” adenoviruses because they need a helper adenovirus that carries essential adenoviral coding regions.

As used herein, the term “gene expression construct” refers to a promoter, at least a fragment of a gene of interest, and a polyadenylation signal sequence. A fully deleted adenoviral vector module of the present disclosure comprises a gene expression construct. A “gene of interest” or “GOI” can be one that exerts its effect at the level of RNA or protein. Examples of genes of interest include, but are not limited to, therapeutic genes, immunomodulatory genes, virus genes, bacterial genes, protein production genes, inhibitory RNAs or proteins, genes coding for products toxic or detrimental to eukaryotic cells or regulatory proteins. For instance, a protein encoded by a therapeutic gene can be employed in the treatment of an inherited disease, e.g., the use of a eDNA encoding the cystic fibrosis transmembrane conductance regulator in the treatment of cystic fibrosis.

Moreover, the therapeutic gene can exert its effect at the level of RNA, for instance, by encoding an antisense message or ribozyme, an siRNA as is known in the art, an alternative RNA splice acceptor or donor, a protein that affects splicing or 3′ processing (e.g., polyadenylation), or a protein that affects the level of expression of another gene within the cell (i.e., where gene expression is broadly considered to include all steps from initiation of transcription through production of a processed protein), perhaps, among other things, by mediating an altered rate of mRNA accumulation, an alteration of mRNA transport, and/or a change in post-transcriptional regulation.

As used herein, the phrase “gene therapy” refers to the transfer of genetic material (e.g., DNA or RNA) of interest into a host to treat or prevent a genetic or acquired disease or condition. The genetic material of interest encodes a product (e.g., a protein polypeptide, peptide or functional RNA) whose production in vivo is desired. For example, the genetic material of interest can encode a hormone, receptor, enzyme or (poly) peptide of therapeutic value. Examples of genetic material of interest include DNA encoding: the cystic fibrosis transmembrane regulator (CFTR), Factor VIII, low density lipoprotein receptor, betagalactosidase, alpha-galactosidase, beta-glucocerebrosidase, insulin, parathyroid hormone, and alpha-1-antitrypsin. Another form of “gene therapy” may entail the delivery of a “toxic” gene to remove unwanted cells or cell populations from the organism for the treatment of, for instance, malignant growths. Examples of such toxic genes are, but not limited to, bacterial L-Methionase, monoclonal antibodies blocking crucial cellular pathways, prodrug converting enzymes, bacterial toxins (Botulinum toxin, Tetanus toxin, Shiga toxin, Diphtheria toxin. Cholera toxin, Diphtheria toxin, Anthraxtoxin LF, listeriolysin), and plant toxins (ricin).

By “gene delivery vector” is meant a composition including an encapsidated fully-deleted adenovirus based vector of the present disclosure packaged without helper adenovirus.

The term “helper-independent” as used herein refers to the process for creating an encapsidated fully deleted adenovirus-based vector module of the present disclosure that does not need the presence of a helper virus for its replication and encapsidation.

Adenovirus includes minimally modified “first-generation” and “second generation” adenoviral vectors. A first-generation adenoviral vector refers to an adenovirus in which exogenous DNA replaces the E1 region, or optionally the E3 region, or optionally both the E1 and E3 region. A second-generation adenovirus vector refers to a first-generation Adenovirus vector, which, in addition to the E1 and E3 regions, contains additional deletions in the E2 region, the E4 region, and any other region of the adenovirus genome, or a combination thereof.

The term “helper virus” as used herein refers to virus used when producing copies of a helper-dependent viral vector which does not have the ability to replicate on its own. The helper virus is used to co-infect cells alongside the gutless virus and provides the necessary enzymes for replication of the genome of the gutless virus and the structural proteins necessary for the assembly of the gutless virus capsid.

The term “inhibitory expression cassette” as used herein refers to a DNA construct consisting of a promoter and a polyadenylation site into which a gene sequence is inserted and expressed that has the ability to inhibit the expression of a gene of interest located on a minimally modified adenoviral vector or a fully deleted adenoviral vector module. This “inhibitory” gene may be homologaus to the gene of interest and antisense to the gene of interest in orientation. The “inhibitory” gene may be coding for a product that interferes with the gene of interest RNA in a different fashion, or may be coding for a product that interferes with the protein encoded by the gene of interest.

The terms “inhibitory RNA fragments” and “inhibitory DNA fragments” as used herein refers to RNA and DNA polynucleotides that have the ability to bind to the RNA of the gene of interest and thus interfere with the expression of the gene of interest. These RNA and DNA fragments may be homologous to the gene of interest and antisense to the gene of interest in orientation. These RNA and DNA fragments may be homologaus to the gene of interest and able to cut the RNA and/or DNA of the gene of interest.

By “immune response” is preferably meant an acquired immune response, such as a cellular or humoral immune response. In the context, of the present specification, an “immunomodulatory molecule” is a polypeptide molecule that modulates, i.e. increases or decreases, a cellular and/or humoral host immune response directed to a target cell in a general or an antigen specific fashion, and preferably is one that decreases the host immune response.

The term “inverted terminal repeat” as used herein refers to DNA sequences located at the left and right termini of the adenovirus genome. These sequences are identical to each other, but placed in opposite directions. The length of the inverted terminal repeats of Adenoviruses vary from about 50 bp to about 170 bp, depending on the serotype of the adenovirus. The inverted terminal repeats contain a number of different cis-acting elements required for viral growth, such as the core origin of viral DNA replication and enhancer elements for the activation of the E1 region.

“In vivo gene therapy” and “in vitro gene therapy” are intended to encompass all past, present and future variations and modifications of what is commonly known and referred to by those of ordinary skill in the art as “gene therapy”, including ex vivo applications.

The term “linear DNA” as used herein refers to non-circularized DNA molecules.

The term “naturally” as used herein refers to as found in nature; wild-type; innately or inherently.

The term “nucleic acid” refers to a deoxyribonucleotide or ribonucleotide polymer in either single- or double-stranded form, and unless otherwise limited, encompasses known analogues having the essential nature of natural nucleotides in that they hybridize to single-stranded nucleic acids in a manner similar to naturally occurring nucleotides (e g., peptide, nucleic acids).

Nucleic acids are “operably linked” when placed into a functional relationship with another nucleic acid sequence. For example, a promoter or enhancer is operably linked to a coding sequence if it affects the transcription of the sequence. Generally, “operably linked” means that the DNA sequences being linked are contiguous. However, enhancers do not have to be contiguous. Linking is accomplished by ligation at convenient restriction sites. If such sites do not exist, the synthetic oligonucleotide adapters or linkers are used in accordance with conventional practice.

The terms “packaging expression plasmid” and “packaging construct” or “pPac” refers to an engineered plasmid construct of circular, double-stranded DNA molecules, wherein the DNA molecules include at least a subset of Adenoviral late genes (e g., L1, L2, L3, L4, L5, E2A, and E4) under control of a promoter. The pPac may be deleted of one or two adenoviral of the inverted terminal repeats (ITRs) and does not include the packaging signal (‘I’}. The pPac is “replication defective”—the viral genome does not comprise sufficient genetic information alone to enable independent replication to produce infectious viral particles within a cell. Any subtype, mixture of subtypes, or chimeric Adenovirus may be used as the source of DNA for generation of the fully deleted adenoviral vector module and the pPac. A pPac may be circular or linear in nature.

The term “packaging signal” as used herein refers to a nucleotide sequence that is present in the virus genome and is necessary for the incorporation of the virus genome inside the virus capsid during virus assembly.

The packaging signal of an adenovirus is naturally located at the left-end terminus down-stream of the left inverted terminal repeat. It may be denoted by “T”.

The term “pathogen” is used in a broad sense to refer to the source of any molecule that elicits an immune response. Thus, pathogens include, but are not limited to, virulent or attenuated viruses, bacteria, fungi, protozoa, parasites, cancer cells and the like. Typically, the immune response is elicited by one or more peptides produced by these pathogens. As described in detail below, genomic DNA encoding the antigenic peptides from these and other pathogens is used to generate an immune response that mirnies the response to natural infection. It will also be apparent in view of the teachings herein, that the methods include the use of genomic DNA obtained from more than one pathogen.

A cell that is “permissive” supports replication of a virus.

The term “plasmid” as used herein refers to an extra-chromosomal DNA molecule separate from the chromosomal DNA which is capable of replicating independently of the chromosomal DNA. In many cases, it is circular and double-stranded.

The term “polylinker” is used for a short stretch of artificially synthesized DNA which carries a number of unique restriction sites allowing the easy insertion of any promoter or DNA segment. The term “heterologous” is used for any combination of DNA sequences that is not normally found intimately associated in nature.

The term “promoter” is intended to mean a regulatory region of DNA that facilitates the transcription of a particular gene. Promoters usually comprise a TATA box capable of directing RNA polymerase II to initiate RNA synthesis at the appropriate transcription initiation site for a particular coding sequence. A promoter may additionally comprise other recognition sequences generally positioned upstream or 5′ to the TATA box, referred to as upstream promoter elements, which influence the transcription initiation rate. A “constitutive promoter” refers to a promoter that allows for continual transcription of its associated gene in many cell types. An “inducible promoter system” refers to a system that uses a regulating agent (including small molecules such as tetracycline, peptide and steroid hormones, neurotransmitters, and environmental factors such as heat, and osmolarity) to induce or to silence a gene. Such systems are “analog” in the sense that their responses are graduated, being dependent on the concentration of the regulating agent. Also, such systems are reversible with the withdrawal of the regulating agent. Activity of these promoters is induced by the presence or absence of biotic or abiotic factors. Inducible promoters are a powerful tool in genetic engineering because the expression of genes operably linked to them can be turned on or off at certain stages of development of an organism or in a particular tissue.

The term “propagate” or “propagated” as used herein refers to reproduce, multiply, or to increase in number, amount or extent by any process.

The term “purification” as used herein refers to the process of purifying or to free from foreign, extraneous, or objectionable elements.

The term “regulatory sequence” (also called “regulatory region” or “regulatory element”) as used herein refers to a promoter, enhancer or other segment of DNA where regulatory proteins such as transcription factors bind preferentially. They control gene expression and thus protein expression.

The term “recombinase” as used herein refers to an enzyme that catalyzes genetic recombination. A recombinase enzyme catalyzes the exchange of short pieces of DNA between two long DNA strands, particularly the exchange of homologous regions between the paired matemal and patemal chromosomes.

The term “restriction enzyme” (or “restriction endonuclease”) refers to an enzyme that cuts double-stranded DNA. The term “restriction sites” or “restriction recognition sites” refer to particular sequences of nucleotides that are recognized by restriction enzymes as sites to cut the DNA molecule. The sites are generally, but not necessarily, palindromic, (because restriction enzymes usually bind as homodimers) and a particular enzyme may cut between two nucleotides within its recognition site, or somewhere nearby.

The term “replication” or “replicating” as used herein refers to making an identical copy of an object such as, for example, but not limited to, a virus particle. The term “replication deficient” as used herein refers to the characteristic of a virus that is unable to replicate in a natural environment. A replication deficient virus is a virus that has been deleted of one or more of the genes that are essential for its replication, such as, for example, but not limited to, the E1 genes. Replication deficient viruses can be propagated in a laboratory in cell lines that express the deleted genes.

The term “stuffer” as used herein refers to a DNA sequence that is inserted into another DNA sequence in order to increase its size. For example, a stuffer fragment can be inserted inside the Adenovirus genome to increase its size to about 36 kb. Stuffer fragments usually do not code for any protein nor contain regulatory elements for gene expression, such as transcriptional enhancers or promoters.

The term “target” or “targeted” as used herein refers to a biological entity, such as, for example, but not limited to, a protein, cell, organ, or nucleic acid, whose activity can be modified by an external stimulus. Depending upon the nature of the stimulus, there may be no direct change in the target, or a conformational change in the target may be induced.

As used herein, a “target cell” can be present as a single entity, or can be part of a larger collection of cells. Such a “larger collection of cells” may comprise, for instance, a cell culture (either mixed or pure), a tissue (e. g., epithelial or other tissue), an organ (e g., heart, lung, liver, gallbladder, urinary bladder, eye or other organ), an organ system (e.g., circulatory system, respiratory system, gastrointestinal system, urinary system, nervous system, integumentary system or other organ system), or an organism (e.g., a bird, mammal, particularly a human, or the like). Preferably, the organs/tissues/cells being targeted are of the circulatory system (e.g., including, but not limited to heart, blood vessels, and blood), respiratory system (e.g., nose, pharynx, larynx, trachea, bronchi, bronchiales, lungs, and the like), gastrointestinal system (e.g., including mouth, pharynx, esophagus, stomach, intestines, salivary glands, pancreas, liver, gallbladder, and others), urinary system (e.g., such as kidneys, ureters, urinary bladder, urethra, and the like), nervous system (e.g, including, but not limited to, brain and spinal cord, and special sense organs, such as the eye) and integumentary system (e.g, skin). Even more preferably, the cells are selected from the group consisting of heart, blood vessel, lung, liver, gallbladder, urinary bladder, eye cells and stem cells. In an embodiment, the target cells are hepatocytes, and a method is provided for veto vector mediated transplantation of allogeneic hepatocytes in a subject. In an embodiment, the target cells are keratinocytes, and a method is provided for veto vector mediated transplantation of allogeneic keratinocytes in a subject, for example, engineered skin. In an embodiment, the target cells are pancreatic islets. In an embodiment, the target cells are cardiomyocytes. In an embodiment, the target cells are kidney cells, and a method is provided for veto vector mediated transplantation of allogeneic kidneys in a subject. In an embodiment, the target cells are fibroblasts, and a method is provided for veto vector mediated transplantation of allogeneic fibroblasts in a subject, for example, engineered skin.

In an embodiment, the target cells are neurons.

In an embodiment, the target cells are glia cells.

The term “transfection” as used herein refers to the introduction into a cell DNA as DNA (for example, introduction of an isolated nucleic acid molecule or a construct of the present disclosure). An adenoviral packaging cell line disclosed herein may be transfected with at least one of a fully deleted adenoviral vector module or a pPac of the present disclosure. An adenoviral packaging cell line disclosed herein may be transfected or transduced with DNA or a viral particle of a minimally modified adenoviral vector.

The term “transduction” as used herein refers to the introduction into a cell DNA either as DNA or by means of a gene transfer vector of the present disclosure. A gene transfer vector of the present disclosure can be transduced into a target cell.

The term “vector” refers to a nucleic acid used in infection of a host cell and into which can be inserted a polynucleotide. Vectors are frequently replicons. Expression vectors permit transcription of a nucleic acid inserted therein. Some common vectors include, but are not limited to, plasmids, cosmids, viruses, phages, recombinant expression cassettes, and transposons. The term “vector” may also refer to an element which aids in the transfer of a gene from one location to another.

The term “viral DNA” as used herein refers to a sequence of DNA that is found in virus particles. The term “viral genome” as used herein refers to the totality of the DNA that is found in virus particles, and that contains all the elements necessary for virus replication. The genome is replicated and transmitted to the virus progeny at each cycle of virus replication.

The term “virions” as used herein refers to a viral particle. Each virion consists of genetic material within a protective protein capsid.

The term “wild-type” as used herein refers to the typical form of an organism, strain, gene, protein, nucleic acid, or characteristic as it occurs in nature. Wild-type refers to the most common phenotype in the natural population.

The terms “wild-type” and “naturally occurring” are used interchangeably.

Fully Deleted Adenoviral Vectors

Fully deleted adenoviral vector module as used herein refers to a linear DNA sequence or a circular sequence from a linear DNA sequence can be released that only contains cis acting adenoviral sequences (i.e. ITRs, \II) necessary for viral replication and encapsidation of the fully adenoviral vector module into a adenoviral capsid. Fully deleted adenoviral vectors only carry the cis acting sequences (i.e. ITRs, W) necessary for viral genome replication and encapsidation. Some systems and methods for packaging fully deleted adenoviral vectors (fully deleted adenoviral vector modules), require their coinfection with an adenoviral “helper” virus, which can be a source of immunogenic adenoviral antigens. Methods have been proposed to remove the contaminating adenoviral helper virus from the therapeutic adenoviral vector preparations. One example is to flank (flax) the packaging site Ψ in the adenoviral helper virus with lox sequences for the Cre recombinase. In theory, passage of the fully deleted adenoviral vector and the “floxed” adenoviral helper virus would decrease contamination by excising the Ψ (packaging) sequence from the adenoviral helper virus and thereby preventing the packaging of the adenoviral helper helper virus. In practice, this approach has not been able to reduce adenoviral helper” virus contamination below 1-in-10³.

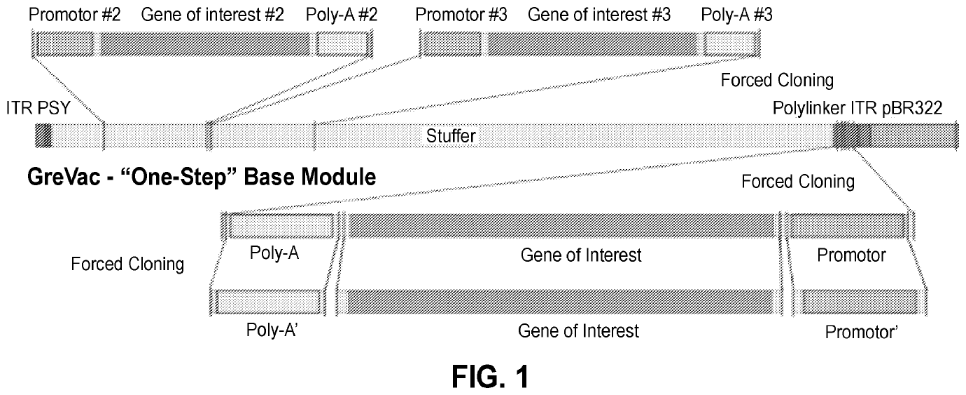

FIG. 1 is a diagrammatic representation showing an embodiment of a design of a fully deleted adenoviral vector module. This fully deleted adenoviral vector module contains cis acting adenoviral sequences (i.e. ITRs, T) necessary for viral replication and encapsidation of the fully adenoviral vector module into an adenoviral capsid together with non-adenoviral stuffer sequences and a polylinker site, into which expression cassettes of single or several genes of interest can be cloned. Addition restriction sites are found within the stuffer, into which addition expression cassettes of single or several genes of interest can be cloned.

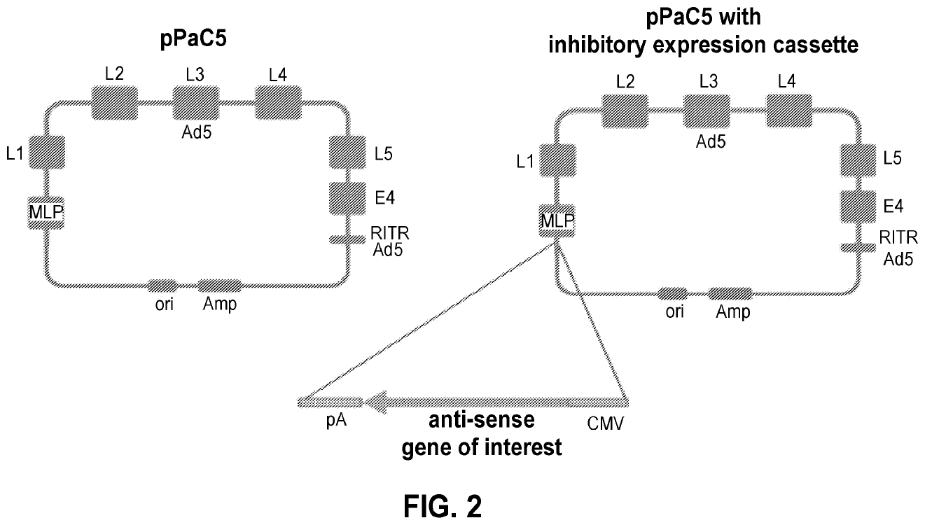

FIG. 2 is a diagrammatic representation showing an embodiment of the design of a packaging expression plasmid with and without expression cassettes able to inhibit expression of genes of interest on the adenoviral vector module. The packaging expression plasmid consists of a plasmid of double stranded DNA that is composed of a subset of adenoviral late genes (e.g., LI, L2, L3, L4, L5, E2A, and E4) under control of a promoter. It may be deleted of one or two adenoviral of the inverted terminal repeats (ITRs) and does not include the packaging signal (Ψ).

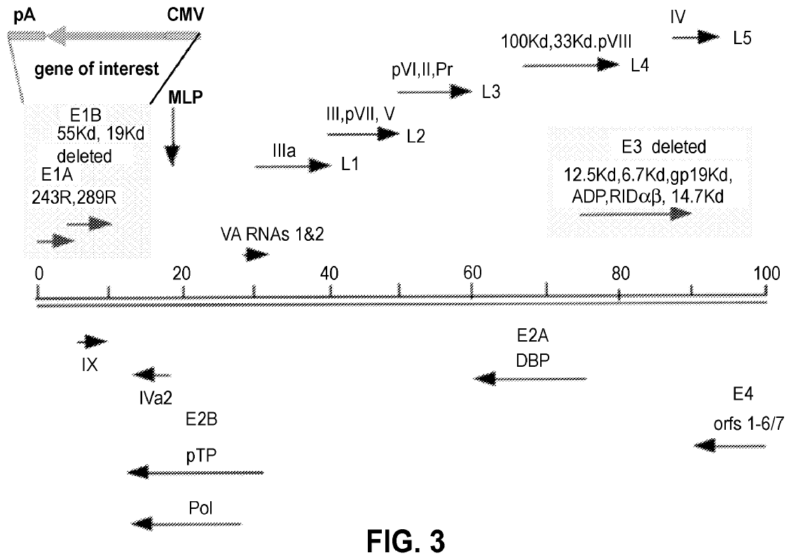

FIG. 3 is a diagrammatic representation showing an embodiment of a minimally modified adenoviral vector that carries a subset of adenoviral late genes (e.g, LI, L2, L3. L4, L5, E2A, and E4) under control of a promoter, as well as two adenoviral inverted repeats (ITRs) and the packaging signal (Ψ).

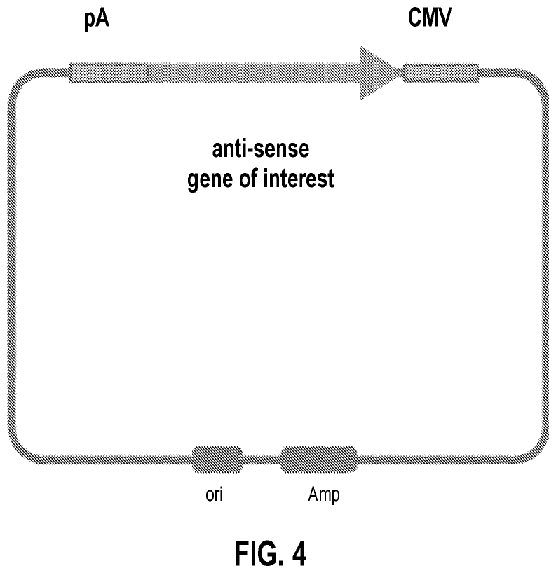

FIG. 4 is a diagrammatic representation showing an embodiment of a design of inhibitory expression plasmid. It carries an expression cassette or expression cassettes able to inhibit expression of a gene or genes of interest found on the adenoviral vector module.

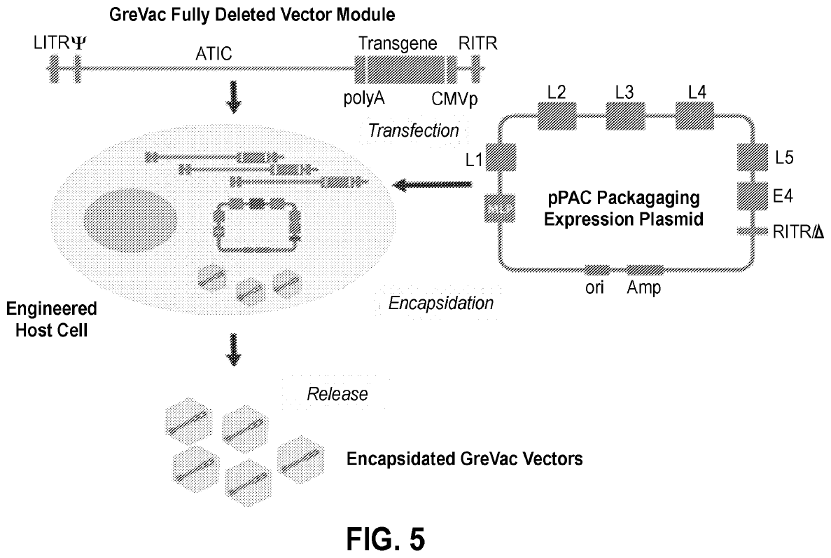

FIG. 5 is a diagrammatic representation showing an embodiment of a method for the packaging by co-transfection of a fully deleted adenoviral vector module with a packaging expression plasmid. Both DNA constructs are mixed at certain molar ratios and co-transfected into a packaging cell, wherein the fully deleted adenoviral vector module is replicated and encapsidated. The genetic program for this process is coded on the packaging expression plasmid. The encapsidated fully deleted adenoviral vector is harvested from the packaging cell.

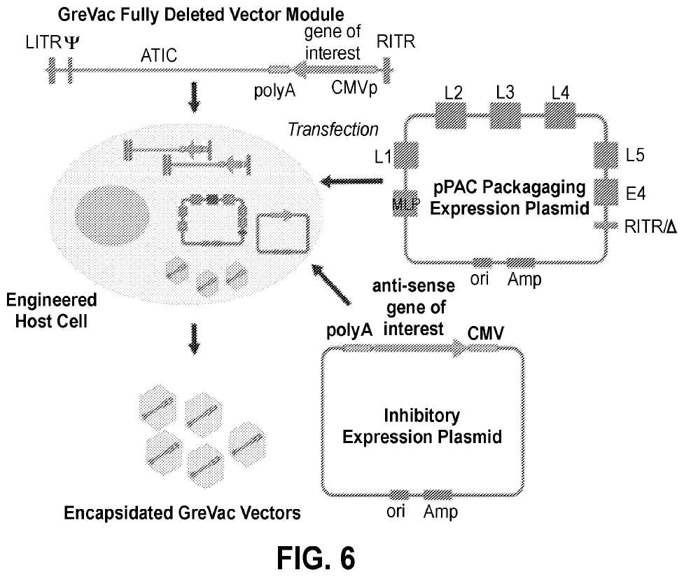

FIG. 6 is a diagrammatic representation showing an embodiment of a method for the encapsidation by co-transfection of a fully deleted adenoviral vector module with a packaging plasmid and an expression plasmid able to inhibit expression of the gene of interest on the adenoviral vector module. The DNA constructs are mixed at certain molar ratios and co-transfected into a packaging cell, wherein the fully deleted adenoviral vector module is replicated and encapsidated. The genetic program for this process is coded on the packaging expression plasmid. Expression of the gene of interest or the genes of interest is inhibited by genes, such as an anti-sense of anti-sense constructs of the gene of interest, expressed on the inhibitory expression plasmid. The encapsidated fully deleted adenoviral vector is harvested from the packaging cell.

Expression of the gene of interest or the genes of interest is inhibited by presence of the RNA or DNA fragments, such as an anti-sense of anti-sense constructs of the gene of interest. The encapsidated fully deleted adenoviral vector is harvested from the packaging cell.

One skilled in the art will appreciate that suitable methods of administering a gene transfer vector of the present disclosure to a human subject or an animal for therapeutic purposes, e.g., gene therapy, immunosuppressive therapy, tissue engineering, vaccination, and the like (see, for example, Rosenfeld et al., Science, 252, 431 434 (1991), Jaffe et al., Clin. Res., 39(2), 302A (1991), Rosenfeld et al., Clin. Res., 39(2), 311A (1991), Berkner, BioTechniques, 6, 616 629 (19SS), are available, and, although more than one route can be used to administer the gene transfer vector, a particular route can provide more immediate and more effective reaction than another administration, rate of excretion, drug combination, the severity of the route. Pharmaceutically acceptable excipients are also well known to those who are skilled in the art, andare readily available. The choice of excipient will be determined in part condition, and the host undergoing therapy.

The dose administered to an animal, particularly a human, in the context of the present invention will vary with the therapeutic transgene of interest and/or the nature of molecule, the composition employed, the method of administration, and the particular site and by the particular method used to administer the gene transfer vector. Accordingly, there is a wide variety of suitable formulations of the gene transfer vectors of the present invention. The following formulations and methods are merely exemplary and are in no way limiting. However, oral, injectable and aerosol formulations are preferred. Formulations suitable for oral administration can consist of a) liquid solutions; (b) capsules, sachets or tablets; (c) suspensions in an appropriate liquid; and (d) suitable emulsions. In an embodiment, the gene transfer vectors of the present invention, alone or in combination with other suitable components, can be made into aerosol formulations to be administered via inhalation. These aerosol formulations can be placed into pressurized acceptable propellants, such as dichlorodifluoromethane, propane, nitrogen, and the like. The gene transfer vectors of the present invention may also be formulated as pharmaceuticals for non-pressured preparations such as in a nebulizer or an atomizer Formulations suitable for parenteral administration include aqueous and non-aqueous, isotonic sterile injection solutions, which can comprise anti-oxidants, buffers, bacteriostats, and solutes that render the formulation isotonic with the blood of the intended recipient, and aqueous and nonaqueous sterile Suspensions that can include suspending agents, solubilizers, thickening agents, stabilizers, and preservatives.

The formulations can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind previously described. The dose administered to an animal, particularly a human, in the context of the present invention will vary with the gene or other sequence of interest, the composition employed, the method of administration, and the particular site and organ that is being treated. The dose should be sufficient to effect a desirable response, e.g., therapeutic or immune response, within a desirable time frame. Hence, one or more of the following routes may administer the gene transfer vectors of the present disclosure: oral administration, injection (such as direct injection), topical, inhalation, parenteral administration, mucosal administration, intramuscular administration, intravenous administration, subcutaneous administration, intraocular administration or transdermal administration. In an embodiment, encapsidated fully deleted adenoviral vectors of the present disclosure are administered via injection. In an embodiment, gene transfer vectors of the present disclosure are administered topically. In an embodiment, gene transfer vectors of the present disclosure are administered by inhalation. In an embodiment, gene transfer vectors of the present disclosure are administered by one or more of: parenteral, mucosal, intramuscular, intravenous, subcutaneous, intraocular or transdermal administration means, and are formulated for such administration. Typically, a physician will determine the actual dosage of gene transfer vectors that will most suitable for an individual subject and it will vary with the age, weight and response of the particular patient and severity of the condition. The specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy. The dose administered to an animal, particularly a human, in the context of the present invention will vary with the therapeutic transgene of interest and/or the nature of the immunomodulatory molecule, the composition employed, the method of administration, and the particular site and organism being treated. However, preferably, a dose corresponding to an effective amount of a GDV is employed. An “effective amount” is one that is sufficient to produce the desired effect in a host, which can be monitored using several end-points known to those skilled in the art. For instance, one desired effect is nucleic acid transfer to a host cell. Such but not limited to, a therapeutic effect (e g., alleviation of some symptom associated with the disease, condition, disorder or syndrome being treated), or by evidence of the transferred gene or coding sequence or its expression within the host (e.g., using the polymerase chain reaction, Northern or hybridizations, or transcription assays to detect the nucleic acid in host cells, or using analysis, antibody-mediated detection, or particularized assays to detect protein or polypeptide encoded by the transferred nucleic acid, or impacted in level or function due to such transfer).

These methods described are by no means all-inclusive, and further methods to suit the specific application will be apparent to the ordinary skilled artisan. In this regard, it should be noted that the response of a host to the introduction of a gene transfer vector can vary depending on the dose of virus administered, the site of delivery, and the genetic makeup of the gene transfer vector as well as the trans gene and the means of inhibiting an immune response.

Generally, to ensure effective transfer of the gene transfer vectors of the present invention, it is preferable that about 1 to about 5,000 copies of the gene transfer vector according to the invention be employed per cell to be contacted, based on an approximate number of cells to be contacted in view of the given route of administration, and it is even more preferable that about 3 to about 300 pfu enter each cell. However, this is merely a general guideline, which by no means precludes use of a higher or lower amount, as might be warranted in a particular application, either in vitro or in vivo. Similarly, the amount of a means of inhibiting an immune response, if in the form of a composition comprising a protein, should be sufficient to inhibit an immune response to the recombinant gene transfer vector comprising the transgene or gene of interest. For example, the actual dose and schedule can vary depending on whether the composition is administered in combination with other pharmaceutical compositions, or depending on interindividual differences in pharmacokinetics, drug disposition, and metabolism. Similarly, amounts can vary in in vitro applications, depending on the particular cell type targeted or the means by which the gene transfer vector is transferred. One skilled in the art easily can make any necessary adjustments in accordance with the necessities of the particular Situation.

The delivery of gene transfer vectors may be accomplished in vitro, ex vivo, as in laboratory procedures for transfecting or transducing cells lines, or in vivo or ex vivo, as in the treatment of certain disease states Once the gene transfer vector has been delivered into the cell the nucleic acid encoding the desired oligonucleotide or polynucleotide sequences may be positioned and expressed at different sites. In certain embodiments, the nucleic acid encoding the construct may be stably integrated into the genome of the cell. This integration may be in the specific location and orientation via homologous recombination (gene replacement) or it may be integrated in recombination (gene replacement) or it may be integrated in random, non-specific location (gene augmentation). In further and preferred embodiments, the nucleic acid may be stably maintained in the cell as a separate, episomal segment of DNA. Such nucleic acid segments or “episomes” encode sequences sufficient to permit maintenance and replication independent of or in synchronization with the host cell cycle. Immunomodulatory genes may encode all or a functional portion of a CD8 polypeptide.

Additional immunomodulatory genes encoding immunomodulatory molecules are contemplated, including, but not limited to, IL-10, TGF-beta, IL-2, IL-12, IL-15, IL-18, IL-4 and GMCSF.

Gene Therapy for Gene and Protein Expression

Gene therapy generally involves the introduction into cells of therapeutic genes, also known as genes of interest or transgenes, whose expression results in amelioration or treatment of genetic disorders. The therapeutic genes involved may be those that encode proteins, structural or enzymatic RNAs, inhibitory products such as antisense RNA or DNA, or any other gene product. Expression is the generation of such a gene product or the resultant effects of the generation of such a gene product. Thus, enhanced expression includes the greater production of any therapeutic gene or the augmentation of that product's role in determining the condition of the cell, tissue, organ or organism.

In general, the instant invention relates to GDV s for transferring selected genetic material of interest (e.g., DNA or RNA) to cells in vivo. The invention also relates to methods of gene therapy using the disclosed gene transfer vectors and genetically engineered cells produced by the method. Diseases that may be treated by the present invention include, but arenot limited to, Hemophilia A (with Factor VIII), Parkinson's Disease, Congestive Heart Failure and Cystic Fibrosis.

In an embodiment, a gene transfer vector of the present disclosure carrying at least a fragment of a gene of interest can infect the myocardium in vivo following intracardiac injection. In an embodiment, a gene transfer vector of the present disclosure carrying at least a fragment of the CFTR gene can be introduced in situ into the lungs of a Cystic Fibrosis patient by aerosol inhalation. In an embodiment, a gene therapy vector of the present disclosure carrying at least a fragment of a gene coding for Factor VIII can be introduced in situ into a muscle in the arm of a patient with Hemophilia A. In an embodiment, a gene transfer vector of the present disclosure carrying at least a fragment of the ADA gene can be transduced into a subpopulation of bone marrow cells ex vivo, and then the transduced bone marrow cells can be transplanted into a patient suffering from Adenosine deaminase (ADA) deficiency. The particular therapeutic gene encoded by a gene therapy vector of the present disclosure is not limiting and includes those useful for various therapeutic and research purposes, as well as reporter genes and reporter gene systems and constructs useful in tracking the expression of transgenes and the effectiveness of Adenoviral vector transduction. Thus, by way of example, the following are classes of possible genes whose expression may be enhanced by using a GDV of the present disclosure: developmental genes (e.g. adhesion molecules, cyclin kinase inhibitors, Wnt family members, Pax family members, Winged helix family members, Hox family members, cytokines/lymphokines and their receptors, growth or differentiation factors and their receptors, neurotransmitters and their receptors), oncogenes (e.g. ABU, BLCI, BCL6, CBFAI, CBL, CSFIR, ERBA, ERBB, EBRB2, ETSI, ETSI, ETV6, FGR, FOX, FYN, HCR, HRAS, JUN, KRAS, LCK, L YN, MDM2, MLL, MYB, MYC, MYCU, MYCN, NRAS, PIMI, PML, RET, SRC, TAU, TCL3 and YES), tumor suppresser genes (e.g. APC, BRCAI, BRCA2, MADH4, MCC, NFI, NF2, R131, TP53 and WTI), enzymes (e g. ACP desaturases and hycroxylases, ADP-glucose pyrophorylases, ATPases, alcohol dehycrogenases, amylases, amyloglucosidases, catalases, cellulases, cyclooxygenases, decarboxylases, dextrinases, esterases, DNA and RNA polymerases, hyaluron synthases, galactosidases, glucanases, glucose oxidases, GTPases, helicases, hemicellulases, hyaluronidases, integrases, invertases, isomersases, kinases, lactases, lipases, lipoxygenases, lyases, lysozymes, pectinesterases, peroxidases, phosphatases, phospholipases, phosphorylases, polygalacturonases, proteinases and peptideases, pullanases, recombinases, reverse transcriptases, topoisomerases, xylanases, reporter genes (e.g. Green fluorescent protein and its many color variants, luciferases, CAT reporter systems, Betagalactosidase, etc.), blood derivatives, hormones, lymphokines (including interleukins), interferons, TNF, growth factors, neurotransmitters or their precursors or synthetic enzymes, trophic factors (such as BDNF, CNTF, NGF, IGF, GMF, aFGF, bFGF, NT3, NTS, and the like), apolipoproteins (such as ApoAI, ApoAIV, ApoE, and the like), dystrophin or a minidystrophic, tumor suppressor genes (such as p53, Rb, RapiA, DCC, k-rev, and the like), genes coding for factors involved in coagulation (such as factors VII, VI, IX, and the like), suicide genes (such as thymidine kinase), cytosine deaminase, or all or part of a natural or artificial immunoglobulin (Fab. ScFv, and the like). Other examples of therapeutic genes include fus, interferon α, interferon ß, interferon γ, and ADP (Adenoviral death protein). The therapeutic gene can also be an antisense gene or sequence whose expression in the target cell enables the expression of cellular genes or the transcription of cellular mRNA to be controlled. Such sequence can, for example, be transcribed in the target cell into RNAs complementary to cellular mRNAs. The therapeutic gene can also be a gene coding for an antigenic peptide capable of generating an immune response in man. In this particular embodiment, the disclosure makes it possible to produce vaccines enabling humans to be immunized, in particular against microorganisms, viruses and cancer. Various enzyme genes are also considered therapeutic genes. Particularly appropriate genes for expression include those genes that are thought to be expressed at less than normal level in the target cells of the subject mammal Examples of particularly useful gene products include, but are not limited to, carbamoyl synthetase I, ornithine transcarbamylase, arginosuccinate synthetase, arginosuccinate lyase, and arginase. Other desirable gene products include fumary-lacetoacetate hydrolase, phenylalanine hydroxylase, alpha-antitrypsin, glucose-6-phosphatase, lowdensity-lipoprotein receptor, porphobilinogen deaminase, factor VIII, factor IX, cystathione .beta.-synthase, branched chain ketoacid decarboxylase, albumin, isovaleryi-CoA dehydrogenase, propionyl CoA carboxylase, methyl malonyl CoA mutase, glutaryl CoA dehydrogenase, insulin, a-glucosidase, pyruvate carboxylase, hepatic phosphorylase, phosphorylase kinase, glycine decarboxylase (also referred to as P-protein), H-protein, T-protein, Menkes disease copper-transporting ATPase, and Wilson's disease copper-transporting ATPase. Other examples of gene products include, but are not limited to, cytosine deaminase, galactose-1-phosphate hypoxanthine-guanine phosphoribosyltransferase, uridyltransferase, phenylalanine hydroxylase, glucocerbrosidase, sphingomyelinase, a-L-iduronidase, glucose-6-phosphate dehydrogenase, HSV thymidine kinase and human thymidine kinase. Hormones are another group of genes that may be used in the Adenoviral-derived vectors described herein. Included are growth hormone, prolactin, placental lactogen, luteinizing hormone, follicle-stimulating hormone, chorionic gonadotropin, thyroidstimulating hormone, leptin, adrenocorticotropin (ACTH}, angiotensin I and II, endorphin, melanocyte stimulating hormone (MSH), cholecystokinin, endothelin, galanin, gastric inhibitory peptide (GIP}, glucagon, insulin, lipotropins, neurophysins, somatostatin, calcitonin, calcitonin gene related peptide (CGRP), calcitonin gene related peptide, hypercalcemia of malignancy factor (1-40), parathyroid hormone-related protein (107-139) (PTH-rP), parathyroid hormone related protein (107-111) (PTH-rP), glucagon-like peptide (GLP-1), pancreastatin, pancreatic peptide, peptide YY, PHM, secretin, vasoactive intestinal peptide (VIP), oxytocin, vasopressin (AVP), vasotocin, enkephalinamide, metorphinamide, alpha melanocyte stimulating hormone (alpha-MSH), atrial natriuretic factor (5-28) (ANF), amylin, amyloid P component (SAP-1), corticotropin releasing hormone (CRH), growth hormone releasing factor (GHRH), luteinizing hormone-releasing hormone (LHRH), neuropeptide Y, substance K (neurokinin A), substance P and thyrotropin releasing hormone (TRH). Other classes of genes that are contemplated to be inserted into the GDVs of the present disclosure include, but are not limited to, interleukins and cytokines, including interleukin 1 (IL-1), IL-2, IL-3, IL4, IL-5, IL-6, IL-7, IL-S, IL-9, IL-10, IL-11 IL-12, GM-CSF and G-CSF.

Diseases that may be treated by the present invention include, but are not limited to, prevalent genetic diseases such as Phenylketonuria (phenylalanine-L-monooxygenase), adenosine deaminase deficiency, cystic fibrosis (cystic fibrosis conductance regulator), Parkinson's disease, ornithine caramyltransferase deficiency (OTC), hemophilias (Factor IX-deficiency, Factor VIII-deficiency), Tay-Sachs (N-acetylhexosamimidase A), cystic fibrosis, which would involve the replacement of the cystic fibrosis conductance regulator gene, and other lipid storage diseases. In addition, the gene encoding erythropoietin (EPO) can used.

In addition, bacterial, plant and eukaryotic toxins are envisioned as transgene. They are, but not limited to Botulinum toxin, Tetanus toxin, Shiga toxin, Diphtheria toxin, Cholera toxin, Diphtheria toxin, Anthrax toxin LF, listeriolysin, and ricin.

A fully deleted adenoviral vector of the present disclosure is free of adenoviral early region and late genes. A gene transfer vector of the present disclosure can be used for treatment of hyper-proliferative disorders such as rheumatoid arthritis or restenosis by transfer of genes encoding angiogenesis inhibitors or cell cycle inhibitors. Transfer of prodrug activators such as the HSVTK gene can be also be used in the treatment of hyperploiferative disorders, including cancer. In an embodiment, a gene transfer vector of the present disclosure includes a therapeutic gene sequence and a CD8 gene sequence, wherein the CD8 gene sequence is capable of preventing an immune response to the therapeutic gene sequence, as described below. Such applications include, but are not limited to, Factor VIII deficiency (Hemophilia A) where the patients do not produce any protein from the deficient gene (null alleles) In these patients, an immune response may be mounted against the product of the therapeutic gene, just as an immune response frequently occurs in Hemophilia A patients treated by injection of Factor VIII protein. Adenovirus vectors have been used to produce recombinant proteins useful in treating diseases. However, contamination of the therapeutic recombinant proteins with adenoviral proteins and/or an adenovirus is a continual challenge. Accordingly, there is a need for systems that support growth of adenoviral-derived with reduced complement of adenoviral genes and/or absence of contamination with replication competent, or helper adenovirus. Co-ordinate expression of polypeptides for multimeric proteins can be coordinated by co-expression from an adenoviral vector. For example, the expression of equimolar amounts of immunoglobulin heavy and light chains is facilitated by co-ordinate expression from an adenoviral vector. According to aspects illustrated herein, in an embodiment a protein expression protocol includes administering a gene transfer vector of the present disclosure.

Vaccine Development

In an embodiment, the invention relates to biotechnology and the development and manufacture of vaccines. The invention is particularly useful for the production of vaccines to aid in protection against viral and bacterial pathogens for vertebrates, in particular mammalians and especially humans.

Vaccines of the present invention can be prophylactic (e.g. to prevent or ameliorate the effects of a future infection by any natural or “wild” pathogen), or therapeutic (e.g. vaccines against cancer). Vaccination is the most important route of dealing with viral infections. Although a number of antiviral agents are available, typically these agents have limited efficacy. Administering antibodies against a virus may be a good way of dealing with viral infections once an individual is infected passive immunization) and typically human or humanized antibodies do seem promising for dealing with a number of viral infections. But the most efficacious and safe way of dealing with virus infection is, and probably will be, prophylaxis through active immunizations. Active immunization is generally referred to as vaccination and vaccines comprising at least one antigenic determinant of a virus, preferably a number of different antigenic determinants of at least one virus, e.g., by incorporating in the vaccine at least one viral polypeptide or protein derived from a virus (subunit vaccines). Typically, the formats mentioned so far include adjuvants in order to enhance an immune response. This also is possible for vaccines based on whole virus, e.g., in an inactivated format. A further possibility is the use of live, but attenuated forms of the pathogenic virus. A further possibility is the use of wild-type virus, e.g., in cases where adult individuals are not in danger from infection, but infants are and may be protected through maternal antibodies and the like. Production of vaccines is not always an easy procedure. In some cases the production of viral material is on eggs, which leads to difficulty in purifying material and extensive safety measures against contamination, etc. Also production on bacteria and or yeasts, which sometimes, but not always, is an alternative for eggs, requires many purification and safety steps. Production on mammalian cells would be an alternative, but mammalian cells used so far all require, for instance, the presence of serum and/or adherence to a solid support fo growth. In the first case, again, purification and safety and e.g., the requirement of protease to support the replication of some viruses become an issue. In the second case, high yields and ease of production become a further issue. Vaccines are still lacking for many viral diseases of great public health importance. Killed viral vaccines can be dangerous and expensive to produce, and are frequently not immunogenic. The inclusion of viral protein encoding sequences in an Adenoviral vector may circumvent these problems, however, there are challenges to creating such an adenovirus vector. For example, there is little space in adenovirus vectors, and the immune response to the adenovirus vector interferes with the immune response to the vaccine protein.

An object of the present invention is therefore to provide gene transfer vectors, which are capable of a long-term maintenance in a large and increasing number of different cells of the host's body and thereby capable of providing a stable expression of the desired antigen(s). Another object of the invention is to provide gene transfer vectors, which are maintained for a long period of time in the cells that originally received the vector and transferred it to the daughter cells after mitotic cell division. Yet another object of the invention is to provide gene transfer vectors, which express in addition to the gene or genes of interest preferably only a gene necessary for a long-term maintenance in the recipient cells and thus are devoid of components that are toxic or cause symptoms of the disease to the recipient. A further object of the invention is to provide gene transfer vectors, which mimic attenuated live viral vaccines, especially in their function of multiplying in the body, without inducing any considerable signs of disease and without expressing undesired proteins, which may induce adverse reactions in a host injected with the DNA vaccine. The vaccines of the present invention comprise a gene transfer vector of the present invention or a mixture of said vectors in a suitable pharmaceutical carrier. The vaccines of the invention are formulated using standard methods of vaccine formulation to produce vaccines to be administered by any conventional route of administration, i.e. intramuscularily, intradermally and like. In specific embodiments, a gene transfer vector of the invention is used to treat and/or prevent an infectious (passive immunization) and typically human or humanized antibodies do seem promising for dealing with a number of viral infections. But the most efficacious and safe way of dealing with virus infection is, and probably will be, prophylaxis through active immunizations. Active immunization is generally referred to as vaccination and vaccines comprising at least one antigenic determinant of a virus, preferably a number of different antigenic determinants of at least one virus, e.g., by incorporating in the vaccine at least one viral polypeptide or protein derived from a virus (subunit vaccines). Typically, the formats mentioned so far include adjuvants in order to enhance an immune response. This also is possible for vaccines based on whole virus, e g., in an inactivated format. A further possibility is the use of live, but attenuated forms of the pathogenic virus. A further possibility is the use of wild-type virus, e.g., in cases where adult individuals are not in danger from infection, but infants are and may be protected through maternal antibodies and the like. Production of vaccines is not always an easy procedure. In some cases the production of viral material is on eggs, which leads to difficulty in purifying material and extensive safety measures against contamination, etc. Also production on bacteria and or yeasts, which sometimes, but not always, is an alternative for eggs, requires many purification and safety steps. Production on mammalian cells would be an alternative, but mammalian cells used so far all require, for instance the presence of serum and/or adherence to a solid support for growth. In the first case, again, purification and safety and e g., the requirement of protease to support the replication of some viruses become an issue. In the second case, high yields and ease of production become a further issue. Vaccines are still lacking for many viral diseases of great public health importance. Killed viral vaccines can be dangerous and expensive to produce, and are frequently not immunogenic. The inclusion of viral protein encoding sequences in an Adenoviral vector may circumvent these problems, however, there are challenges to creating such an adenovirus vector. For example, there is little space in Adenovirus vectors, and the immune response to the adenovirus vector interferes with the immune response to the vaccine protein. An object of the present invention is therefore to provide gene transfer vectors, which are capable of a long-term maintenance in a large and increasing number of different cells of the host's body and thereby capable of providing a stable expression of the desired antigen(s). Another object of the invention is to provide gene transfer vectors, which are maintained for a long period of time in the cells that originally received the vector and transferred it to the daughter cells after mitotic cell division. Yet another object of the invention is to provide gene transfer vectors, which express in addition to the gene or genes of interest preferably only a gene necessary for a long-term maintenance in the recipient cells and thus are devoid of components that are toxic or cause symptoms of the disease to the recipient. A further object of the invention is to provide gene transfer vectors, which mimic attenuated live viral vaccines, especially in their function of multiplying in the body, without inducing any considerable signs of disease and without expressing undesired proteins, which may induce adverse reactions in a host injected with the DNA vaccine. The vaccines of the present invention comprise a gene transfer vector of the present invention or a mixture of said vectors in a suitable pharmaceutical carrier. The vaccines of the invention are formulated using standard methods of vaccine formulation to produce vaccines to be administered by any conventional route of administration, i.e. intramuscularly, intradermally and like. In specific embodiments, a gene transfer vector of the invention is used to treat and/or prevent an infectious disease.

A gene transfer vector of the present disclosure can be used for vaccine development to protect an individual against a disease by inducing immunity. One advantage of using a GDV of the present disclosure for vaccine development is that the recipient's immune response is not deflected by Ad genes. In an embodiment, a gene transfer vector of the present disclosure encodes one or more proteins and/or RNAs (antigens) from viruses of importance for human health or agriculture. In an embodiment, the vaccine is used to protect an individual against a disease by inducing immunity. In an embodiment, multiple genes of interest my be included for multivalent vaccines, from the same or different pathogens. In certain embodiments, the gene transfer vector further comprises one or more expression cassettes of a DNA sequence of interest. In certain, the DNA sequence of interest is that of an infectious pathogen. In certain embodiments, the infectious pathogen is a virus.

In certain specific embodiments, the virus is selected from the group consisting of Human Immunodeficiency Virus (HIV), Herpex Simplex Virus (HSV), Hepatitis C Virus, Influenzae Virus, Rotavius, Papilloma Virus, Lentivirus, Enterevirus or combinations thereof. In certain embodiments, the DNA sequence of interest is that of a bacterium. In certain embodiments, the bacterium is selected from the group consisting of Chlamydia trachomatis, Mycobacterium tuberculosis, and Mycoplasma pneumonia. In certain embodiments, the DNA sequence of interest is that of a fungal pathogen. In certain embodiments, the DNA sequence of is of HIV origin.

In an embodiment, the vaccine is used to protect an individual against influenza virus. In an embodiment, the influenza virus is swine flu. In an embodiment, the influenza virus is avian flu. In an embodiment, the one or more proteins and/or RNAs of a gene transfer vector of the present disclosure are selected from one of hemagglutinin (HA), neuraminidase (NA), nucleocapsid (NP), M1 (matrix protein), M2 (ion channel), NS 1, NS2 40 (NEP), lipid bilayer, PBI, PB2 or PA. Representative viral and bacterial candidates for vaccines of the present disclosure are listed below. Genes for these vaccines are illustrated in brackets:

### Viruses-Orthomyxoviruses

Influenza A—hemagglutinin (HA) and neuraminidase (NA), nucleoprotein (NP),

### Mu M2, NSI, NS2 (NEP), PA, PB/, PB1-F2 and PB2)

### Influenza B

### Influenza C

### Viruses-Herpes Virus

Herpes simplex 1 (oral herpes), Herpessimplex 2 (genital herpes)—polypeptides; viral glycoproteins (designated gB, gC, gD, gE, gG, gH, gi, gJ, gK, gL, and gM) are known, and another is (gN) predicted; glycoproteins Band 0.

Epstein-Barr (mononucleosis, 8urkitt's Iymphoma, nasopharyngeal carcinoma)—Epstein-Barr nuclear antigen [EBNA] 1, 2, 3A, 38, 3C, LP, and LMP; gp3501220 aka gp340

Cytomegalovirus—G/ycoprotein B, 1 EI, pp 89, gB and pp 65 are the minimum requirements in a vaccine to induce neutralizing antibodies and cytotoxic T lymphocyte (CTL) responses.
Immunisation with additional proteins, e.g., gH, gN for neutralising antibodies and E1, exon 4 and pp 150 for CTL responses, would strengthen protective immune responses.
Varicella zoster virus (chicken pox and shingles)—recombinant proteins from gE, gI and gB genes
Kaposi's sarcoma-associated herpesvirus 8 (Kaposi's sarcoma)

### Herpes 6 (A and 8)

### Herpes 7

Herpes 8—glycoprotein B (gB);
Viruses-Papilloma virus:
For all HPV L 1 capsid protein, E1, E2, E6, and E7 genes
HPV (Cervical carcinoma High-risk. 16, 18, 31, 33, 35, 39, 45, 51, 52, 56, 58, 59,
Probably high-risk: 26, 53, 66, 68, 73, 82)
HPV (common warts: 2, 7)
HPV (Plantar warts: 1, 2, 4, 63)
HPV (Fiat warts: 3, 1 0)
HPV (Anogenital warts: 6, 11, 42, 43, 44, 55)

### Viruses-Reoviridae

Rotavirus A (gastroenteritis)—VP2 and VP6 profeins

### Viruses-Coronaviruses

Severe acute respiratory syndrome coronavirus (Severe Acute Respiratory Syndrome)—SARS-CoV and MERS-CoV are enveloped plus-stranded RNA viruses with a −30 kb genome encoding replicase (Rep) and the structural proteins spike (S), envelope (E), membrane (M), and nucleocapsid (N) spike or nucleocapsid proteins, S and N genes respectively—Human coronavirus 229Espike and envelope genes

### Human Coronavirus NL63

Viruses-Astrovirus (gastroenteritis)—the astrovirus 87-kDa structural polyprotein
Viruses-Norovirus (gastroenteritis)—Viral capsid genes, VPI and VP2

### Viruses-Fiaviviridae

Dengue fever—premembrane (prM) and envelope (E) genes
Japanese encephalitis—prM, E and NSI genes; prM, and envelope (E) coding regions of JE virus.
Kyasanur Forest disease
Murray Valley encephalitis
St. Louis encephalitis
Tick-borne encephalitis
West Nile encephalitis
Hepatitis C—Hepatitis C Virus Glycoprotein E2; glycoproteins E1 and E2 of hepatitis C; The core gene of HCV

### Viruses-Picornaviridae-Enterovirus

Human enterovirus A (21 types including some coxsackie A viruses)
Human enterovirus B (57 types including enteroviruses, coxsackie B viruses)
Human enterovirus C (14 types including some coxsackie A viruses)
Human enterovirus 0 (three types: EV-68, EV-70, EV-94)—VPI gene;

### Viruses-Picornaviridae-Rhinovirus

Human rhinovirus A (74 serotypes)
Human rhinovirus B (25 serotypes)
Human rhinovirus C (7 serotypes)—rhinovirus-derived VPI; the surface protein which is critically involved in infection of respiratory cells

### Viruses-Picornaviridae-Hepatovirus

### Hepatitis A

### Viruses-Togaviridae-Aiphavirus

Sindbis virus
Eastern equine encephalitis virus
Western equine encephalitis virus
Venezuelan equine encephalitis virus
Ross River virus
O'nyong'nyong virus

### Viruses-Togaviridae-Rubivirus

Rubella virus

### Viruses-Togaviridae-Hepevirus

Hepatitis E virus—the ORF2 protein; recombinant HEY capsid protein; the vaccine peptide has a 26 amino acids extension from the N terminal of another peptide, E2, of the HEY capsid protein

### Viruses-Togaviridae-Bomaviridae

Barna disease virus—BDV nucleoprotein (BDV-N)

### Viruses-Togaviridae-Filoviridae

### Ebolavirus

### Marburgvirus

### Viruses-Togaviridae-Paramyxoviruses

### Measles

Sendai virus
Human parainfluenza viruses 1 and 3,

### Mumpsvirus

Humanparainfluenza viruses 2 and 4
Human respiratory syncytial virus
Newcastle disease virus

### Viruses-Togaviridae-Retrovirus

HIV-gag: p18, p24, p55, pol: p31, p51, p66; env: p41, p120,
p160
Hepatitis B virus

### HTLVI, II

### Viruses-Togaviridae-Rhabdoviruses

### Rabies

### Viruses-Togaviridae-Arenaviruses

Ranta virus
Korean hemorrhagic fever
Lymphocytic choriomeningitis virus

### Junin

### Machupo

### Lass a

### Sabia

### Guanarito

California encephalitis
Congo-Crimean hemorrhagic fever
Ritt valley fever

### Viruses-Parvoviruses

Human parvovirus (B 19)

### Bacteria

### Bartonella

Brucella including B. abortus, B. canis, B. suis
Burkholderia (Pseudomonas) mallei, B. pseudomallei
Coxiella burnetii
Francisella tularensis
Mycobacterium bovis (except BCG strain, see Appendix B-11-A, Risk Group 2 (RG2)—Bacterial Agents Including Chlamydia), M. tuberculosis
Pasteurella multocida type B-“buffalo” and other virulent strains
Rickettsia akari, R. australis, R. canada, R. conorii, R. prowazekii, R. rickettsii, R. siberica, R. tsutsugamushi, R. typhi (R. mooseri)
Yersinia pestis

A method of regulating an immune response in an individual includes administering a gene transfer vector of the present disclosure to the individual, wherein the gene transfer vector encodes at least one therapeutic gene (gene of interest, “(GOI”).

### Vectors

Besides gene transfer vectors based on adenoviruses, other vector systems are envisioned whose assemblies are impeded by the production of proteins encoded by transgenes within the vector. Such gene transfer vectors belong to the group of viral vectors, such as, but not limited to, retrovirus vectors, lentivirus vectors, adeno associated virus vectors, SV40-based vectors, vaccinia virus vectors, and Epstain-Barr virus vectors.

The present invention is described in the following examples, which are set forth to aid in the understanding of the invention, and should not be construed to limit in any way follow thereafter. The following examples are put forth so as to provide those of ordinary skill in the art with a complete disclosure and description of how to make and use the described invention, and are not intended to limit the scope of what the inventors regard as their invention nor are they intended to represent that the experiments below are all or the only experiments performed. Efforts have been made to ensure accuracy with respect to numbers used (e.g amounts, temperature, etc.), but some experimental errors and deviations should be accounted for. Unless indicated otherwise, parts are parts by weight, molecular weight is weight average molecular weight, temperature is in degrees Centigrade, and pressure is at or near atmospheric.

The examples should, of course, be understood to be merely illustrative of only certain embodiments of the invention and not to constitute limitations upon the scope of the invention which is defined by the claims that are appended at the end of this description.

Having described the disclosure in detail, it will be apparent that modifications and variations are possible without departing from the scope of the claims.

### EXAMPLES

The following non-limiting examples are provided to further illustrate the present disclosure.

### Example 1

Host cells, such as human embryonal kidney cells, are seeded into tissue culture flasks in eurkaryotic tissue culture medium. They are cultured for three days under CO₂ (5%) and atmospheric oxygen tension. A mixture of the following DNA constructs is added at certain ratio together with a transfectant, such as but not limited to, calcium phosphate: a) a linear fully deleted adenoviral vector module that carries the Ebola glycoprotein gene in expression cassette driven from the CMV promoter, b) a packaging expression plasmid; c) an inhibitory expression plasmid that carries an anti-sense construct of the Ebola glycoprotein driven from a CMV promoter. After three days of culture, the host cells are harvested and lysed by freeze-thawing to release the encapsidated fully deleted adenoviral vector.

When introducing elements of the present disclosure or the preferred embodiment(s) thereof, the articles “a”, “an”, “the”, and “said” are intended to mean that there are one or more of the elements. The terms “comprising”, “including”, and “having” are intended to be inclusive and mean that there may be additional elements other than the listed elements.

In view of the above, it will be seen that the several objects of the disclosure are achieved and other advantageous results attained.

As various changes could be made in the above products and methods without departing from the scope of the disclosure, it is intended that all matter contained in the above description shall be interpreted as illustrative and not in a limiting sense.

1. A method for propagating a fully deleted adenoviral based gene transfer vector comprising: (a) providing an adenovirus packaging cell line: (b) transfecting, into the cell line, a fully deleted adenoviral vector module whose construct includes both adenoviral inverted terminal repeats, the packaging signal, and at least one or more DNA inserts which comprise a gene sequence or gene sequences encoding a protein of interest or proteins of interest, but no adenoviral structural genes: (c) transfecting, into the cell line, a replication defective circular packaging expression plasmid having a subset of adenoviral late genes including L1, L2, L3, L4, L5, E2A, and E4, and a packaging signal, wherein the fully-deleted adenoviral vector construct and the packaging construct can transfect the adenovirus packaging cell line resulting in the encapsidation of a fully deleted adenovirus based gene transfer vector independent of helper Adenovirus vector; and (d) transfecting, into the cell line, an inhibitory expression vector that carries one or more expression cassettes that code for an anti-sense construct or anti-sense constructs of the gene sequence encoding a protein of interest or proteins of interest on the fully deleted adenoviral vector module.

2. The method in claim 1, wherein the fully deleted adenoviral vector module is packaged into an adenoviral capsid without the help of an adenoviral helper virus.

3. The method in claim 1, wherein the fully deleted adenoviral vector carrying a gene of interest or genes of interest with function detrimental or toxic to the host cell, is packaged into an adenoviral capsid.

4. A method for propagating a fully deleted adenoviral based gene transfer vector comprising: (a) providing an adenovirus packaging cell line; (b) transfecting, into the cell line, a fully deleted adenoviral vector module whose construct includes both adenoviral inverted terminal repeats, the packaging signal, and at least one or more DNA inserts which comprise a gene sequence gene sequences encoding a protein of interest or proteins of interest, but no adenoviral structural genes; (c) transfecting, into the cell line, a replication defective circular packaging expression plasmid having a subset of adenovirallate genes including L1, L2, L3, L4, L5 E2A, and E4, and a packaging signal, wherein the fully-deleted adenoviral vector construct and the packaging construct can transfect the adenovirus packaging cell line resulting in the encapsidation of a fully deleted adenovirus based gene transfer vector independent of helper Adenovirus vector, and (d) transfecting, into the cell line, RNA fragments anti-sense of the gene of interest or genes of interest on the fully deleted adenoviral vector module.

5. The method in claim 4, wherein the fully deleted adenoviral vector module is packaging into an adenoviral capsid without the help of an adenoviral helper virus.

6. The method in claim 4, wherein the fully deleted adenoviral vector a gene of interest or genes of interest with function detrimental or toxic to the host cell, is packaged into an adenoviral capsid

7. A method for propagating a fully deleted adenoviral based gene transfer vector comprising: (a) providing an adenovirus packaging cell line; (b) transfecting, into the cell line, a fully deleted adenoviral vector module whose construct includes both adenoviral inverted terminal repeats, the packaging signals, and at least one or more DNA inserts which comprise a gene sequence gene sequences encoding a protein of interest or proteins of interest, but no adenoviral structural genes; (c) transfecting, into the cell line, a replication defective circular packaging expression plasmid having a subset of adenovirallate genes including L1, L2, L3, L4, L5, E2A, and E4, and a packaging signal. wherein the fully-deleted adenoviral vector construct and the packaging construct can transfect the adenovirus packaging cell line resulting in the encapsidation of a fully deleted adenovirus based gene transfer vector independent of helper Adenovirus vector; and (d) transfecting, into the cell line, DNA fragments anti-sense of the gene of interest or genes of interest on the fully deleted adenoviral vector module.

8. The method in claim 7, wherein the fully deleted adenoviral vector module is packaged into an adenoviral capsid without the help of an adenoviral helper virus.

9. The method in claim 7, wherein the fully deleted adenoviral vector module carrying a gene of interest or genes of interest with function detrimental or toxic to the host cell, is packaged into an adenoviral capsid.

10. A method for propagating a fully deleted adenoviral based gene transfer vector comprising: (a) providing an adenovirus packaging cell line; (b) transducing, into the cell line, an encapsidated fully deleted Adenoviral vector whose construct includes both adenoviral inverted terminal repeats, the packaging signal, and at least one or more DNA inserts which comprise a gene sequence gene sequences encoding a protein of interest or proteins of interest, but no adenoviral structural genes; (c) transfecting. into the cell line, a replication defective circular packaging expression plasmid having a subset of adenoviral late, genes including L1 L2, L3, L4, L5, E2A, and E4, and a packaging signal, wherein the fully-deleted adenoviral vector construct and the packaging construct can transfect the adenovirus packaging cell line resulting in the encapsidation of a fully deleted adenovirus based gene transfer vector independent of helper Adenovirus vector, and (d) transfecting, into the cell line, an inhibitory expression vector that carries an or more expression cassette that code for an anti-sense construct of the gene sequence encoding a protein of interest or proteins of interest.

11. The method in claim 10, wherein the deleted adenoviral vector module is packaged into an adenoviral capsid without the help of an adenoviral helper virus.

12. The method in claim 10, wherein the fully deleted adenoviral vector module carrying a gene of interest or genes of interest with function detrimental or toxic to the host cell, is packaged into an adenoviral capsid.

13. A method for propagating a fully deleted adenoviral based gene transfer vector comprising: (a) providing an adenovirus packaging cell line; (b) transducing, into the cell line, an encapsidated fully deleted Adenoviral vector whose construct includes both adenoviral inverted terminal repeats, the packaging signal, and at least one or more DNA inserts which comprise a gene sequence gene sequences encoding a protein of interest or proteins of interest, but no adenoviral structural genes, (c) transfecting, into the cell line, a replication defective circular packaging expression plasmid having a subset of adenovirallate genes including L1, L2, L3, L4, L5, E2A, and E4, and a packaging signal, wherein the full y-deleted adenoviral vector construct and the packaging construct can transfect the adenovirus packaging cell line resulting in the encapsidation of a fully deleted adenovirus based gene transfer vector independent of helper adenovirus vector; and (d) transfecting, into the cell line, fragments anti-sense of the gene of interest or genes of interest on the fully deleted adenoviral vector module.

14. The method in claim 13, wherein the fully deleted adenoviral vector module is packaged into an adenoviral capsid without the help of an adenoviral helper virus.

15. The method in claim 13, wherein the deleted adenoviral vector a gene of interest or genes of interest with function detrimental or toxic to the host cell, is packaged into an adenoviral capsid.

16. A method for propagating a fully deleted adenoviral based gene transfer vector comprising: (a) providing an adenovivus packaging cell line; (b) transducing, into the cell line, an encapsidated fully deleted Adenoviral vector module whose construct includes both adenoviral inverted terminal repeats, the packaging signal, and at least one or more DNA inserts which comprise a gene sequence gene sequences encoding a protein of interest or proteins of interest, but no adenoviral structural genes; (c) transfecting, into the cell line, a replication defective circular packaging expression plasmid having a subset of adenovirallate genes including L1, L2, L3, L4, L5, E2A, and E4, and a packaging signal, wherein the fully-deleted adenoviral vector construct and the packaging construct can transfect the adenovirus packaging cell line resulting in the encapsidation of a fully deleted adenovirus based gene transfer vector independent or helper adenovirus vector; and (d) transfecting, into the cell line, DNA fragments anti-sense of the gene of interest or genes of interest on the fully deleted adenoviral vector module.

17. The method in claim 16, wherein the fully deleted adenoviral vector module is packaged into an adenoviral capsid without the help of an adenoviral helper virus.

18. The method in claim 16, wherein the fully deleted adenoviral vector module carrying a gene of interest or genes of interest with function detrimental or toxic to the host cell, is packaged into an adenoviral capsid.

19-74. (canceled)
